(19) United States
(12) Patent Application Publication
(10) Pub. No.: US 2007/0224702 A1
(43) Pub. Date: Sep. 27, 2007

(54) Flex Method

(75) Inventors: Mats Inganas, Uppsala (SE); Mats Holmquist, Sollentuna (SE)

Correspondence Address: FULBRIGHT & JAWORSKI, LLP, 1301 MCKINNEY, SUITE 5100, HOUSTON, TX, 77010-3095, US

(73) Assignee: GYROS Patent AB, Uppsale (SE)

(21) Appl. No.: 11/689,702

(22) Filed: Mar. 22, 2007

Related U.S. Application Data
(60) Provisional application No. 60/784,604, filed on Mar. 22, 2006.

(51) Int. Cl. G01N 33/558

(52) U.S. Cl. 436/514

(57) ABSTRACT

A method for quantifying n different analytes that are present in one or more liquid samples by performing n different affinity assay formats, each of which is dedicated for a particular one of the analytes. The characteristic feature is that each format is performed in a separate microchannel structures of a microfluidic device that contains at least n microchannel structures, and comprises formation and measurement of an immobilized product (affinity complex) that is formed on a solid phase that is placed in a microcavity of the microchannel structure used for the format in order to quantify the analyte to which the format is dedicated.

### CROSS-REFERENCE TO RELATED APPLICATIONS

This application claims priority to U.S. Provisional Application No. 60/784,604 filed Mar. 22, 2006, which is incorporated herein by reference in its entirety.

### TECHNICAL FIELD

The invention is a method for quantifying the amounts of different analytes (Ans) having the same origin, e.g. be present in the same liquid sample or in samples having different combinations of the analytes (analyte sample(s)). Each of the different analytes is assayed by a format of an affinity assay (ligand-receptor assay) in which an affinity complex is formed (product) in an amount that reflects the amount of analyte in the sample.

### BACKGROUND OF THE INVENTION

When a doctor meets a patient for the first time he is confronted with patient history and possibly symptoms that can be further examined as input to the diagnostic procedure. The diagnostic procedure is greatly simplified by laboratory investigations to support either of several potential diagnoses.

In decentralized diagnostic settings focus is put on generating information that might be helpful in decision-making. In the diagnoses of life-threatening diseases that require immediate medical intervention or when time-efficient analysis procedures are employed for administrative reasons, e.g. to rapidly generate diagnostic information from molecules carrying diagnostic information (biomarkers) found in patients for appropriate diagnosis and prescription of treatment. Traditionally, the combined results of testing for several biomarkers are used, e.g. for AMI (myoglobin, CKMB, troponin I or troponin T), allergy diagnosis (IgE of various specificities) or drug abuse (small molecules with stimulatory effects). Useful markers at this stage of the diagnostic procedure are often disparate with respect to structure, concentrations etc and therefore require different assay formats for quantification. It is therefore often a prerequisite to divert samples from a patient into a number of different process streams typically with at least one process stream for each format and/analyte. In centralized settings there are often means available for taking care of problems associated with several process streams. When a similar situation occurs in a decentralized setting it becomes more complicated from an assay point of view to promptly perform assays and take care of results.

There are also a number of other situations where there often exists a desire to assay disparate analytes that require different assay formats, e.g. clinical studies, animal studies, screening of compound libraries etc.

Microfluidic devices adapted to immunoassays, cell based assays, nucleic acid assays, enzymatic assays, and other kinds of assays utilizing affinity reactions have during the last two decades been considered as valuable tools for performing diagnostic procedures and other kinds of investigations within life science. Each device typically has contained a plurality of microchannel structures that functionally are essentially equal and therefore suitable for performing a dedicated assay format a large number of times—either simultaneously or at different occasions. Microfluidic devices are available in which one “on demand” can functionalize each microchannel structure with the appropriate immune reagent for a given analyte and assay format. A generic way of functionalization has been achieved by the use of a pre-introduced generic ligand, such as streptavidin, that is immobilized to a solid phase, for instance in the form of beads that are placed in a microcavity of each microchannel structure. By introducing a reagent conjugated to the affinity counterpart of the generic ligand into each structure it is easy to introduce in principle any kind of reagent on the solid phase. In real life this kind of systems has primarily been used for carrying out the same assay format in all the microchannel structures of a device, typically the sandwich format. See for instance WO 2004083109 (Gyros AB), WO 200483108 (Gyros AB), PCT/SE06/000071 (Gyros Patent AB) and PCT/SE06/000072 (Gyros Patent AB). It has also been suggested to assay different analytes in the same microchannel structure and/or in different microchannel structure of the same microfluidic device.

### BRIEF SUMMARY OF THE INVENTION

The invention is a method for quantification of the amount of each of a plurality of n different analytes by the use of a plurality of n different formats of affinity assays. Each of the different formats is dedicated for a particular one of the n analytes. The n different analytes may be present in a common liquid sample (analyte sample) or as different combinations of at least one of the analytes in several samples (analyte samples). The affinity reactions of each of the n format and for each of the n analytes will lead to the formation of an affinity complex (=product) in an amount that is a function of the amount of analyte in the sample, i.e. format^{k} will be used for analyte^{k} and result in produce^{k}. The characteristic feature of the innovative method is:
A) all of the plurality of analytes is quantified in the same microfluidic device that comprises n microchannel structures with a separate microchannel structure being used for each of the n formats, and
B) each of the n formats comprises the steps of:
   (i) providing in a microcavity of the microchannel structure used for the format a solid phase that exposes:
      an immobilized capturer^{k} that is an affinity reactant in the format, or
      a) an immobilizing group that is capable of firmly attaching an immobilizable capturer^{k} that is an affinity reactant in the format and exhibits an immobilizing tag that is reactive with the immobilizing group,
   (ii) forming an immobilized form of product^{k} within the microcavity by
      a) performing the affinity reaction(s) of the format to incorporate immobilized capturer^{k} provided in step (i.a) into product^{k}, or
      b) performing the affinity reaction(s) of the format to incorporate immobilizable capturer^{k} into an immobilizable form of produce^{k} and subsequently firmly attaching the product to the solid phase by reacting the immobilizing tag and the immobilizing group of the solid phase provided in step (i.b) with each other, and
   (iii) determining the amount of analyte^{k} in the sample by measuring the amount of immobilized product^{k} formed in step (ii).

In preferred assay formats there may also be used a detectable reactant in order to facilitate the measurement in step (iii). A detectable reactant is typically capable of undergoing an affinity reaction with immobilized or immobilizable capturer^{k} and/or with analyte^{k}, for instance by being an affinity counterpart to capturer^{k} or an affinity counterpart to analyte respectively.

In addition to the analyte¹, analyte² . . . analyteⁿ, the sample(s) may also contain other analytes that are assayed in the same microchannel structures as the n analytes and/or in other microchannel structures which may be located within or outside the microfluidic device. These other analytes may be assayed according to an assay format of the kind used for one of the analyte¹, analyte² . . . analyteⁿ but may also be assayed according to other formats.

Each of one or more of the n different analytes may also be quantified by an alternative format, for instance selected amongst the n formats used for the n different analytes.

The invention requires at least n microchannel structures on the microfluidic device for performing the n formats. These microchannel structures are typically functionally equal by which is meant that every one of the n formats can be carried out in every structure, i.e. everyone of the n structures contains at least the sequence of functionalities that are required by the formats to be carried out according to the invention, for instance selected amongst inlet ports, volume-defining units (for liquid), distribution manifolds (for liquids), mixing units, reaction microcavities, detection microcavities, liquid routers, waste units etc. The term “functionally equal” includes that one or more of the n microchannel structures may be adapted for processing liquid samples of certain volumes (analyte samples, samples of washing liquid, reagent samples etc) while other microchannel structures are adapted for other volumes.

In preferred variants of the invention, the number n of different analytes/formats are two, three, four, five or more, for instance with all of the n formats being one-step formats or two-step formats with preference for the latter that advantageously also is of the forward type, i.e. formats that as the first step comprise incubation of solid phase, such as a porous bed, that exhibits the capturer with a soluble reactant, such as the analyte or the detectable reactant, of the particular format contemplated. Thus in one preferred variant the first step means that the analyte is incubated with the capturer in immobilized form and the second step that the immobilized product formed in the first step is incubated with detectable reactant. Further particularly advantageous embodiments comprises that one, two or more of the two-steps or one-step formats performed according to the invention on the same device are inhibition formats and/or one, two or more of the other two-step formats or other one-step formats are non-inhibition formats, such as sandwich formats. One, two, three or more of these sandwich formats, if included, are in preferred variants different from the others by utilizing one of the reactant combinations A-C that is not used by the others:

A) one of the two analyte counterparts is an antigen/hapten while the other is an antibody relative to the analyte (the analyte is an antigen specific antibody of a certain class, subclass or species),

B) each of the two analyte counterparts area is an antigen relative to the analyte (the analyte is an antigen specific antibody),

C) both of the two analyte counterparts are antibodies of the same or different specificities relative to the analyte (the analyte is a protein in general including for instance IgG, IgE, transferrin, and other multiepitopic biomolecules of high molecular weight and structures as given below for reactants in general).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows an exemplary set of microchannel structures.

### DETAILED DESCRIPTION OF THE INVENTION

### I. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “analyte” includes “analyte-related entities” that have been obtained by processing a liquid sample containing the analyte in such a manner that an original or a native analyte is transformed to an entity different from an original/native analyte and in an amount that is a function of the amount of the original/native analyte in an original/native sample. This kind of processing may take place within and/or outside the microchannel structure/microfluidic device in which the method of the instant invention is carried out. Typical analyte-related entities are affinity complexes that are formed upstream of the microcavity containing the solid phase in an amount related to the amount of original analyte. Such complexes may or may not contain the original/native analyte. Upstream in this context includes outside of and/or within the microfluidic device/microchannel structure.

In this specification an analyte analogue (An-analogue) is a reactant that is different from an analyte but capable of inhibiting an affinity reaction between the analyte and an affinity counterpart to the analyte. If not otherwise indicated “counterpart” refers to affinity counterparts. The prefix “anti” will be used for counterparts that are used as antibodies in the invention, for instance anti-analyte or anti-analyte antibody (anti-An, anti-An antibody), anti-capturer or anti-capturer antibody etc. A counterpart to an analyte is typically also a counterpart to an analyte analogue if the latter is used in the format. The term “antibody” include various kinds of antigen/hapten-binding fragments and derivatives of antibodies as well as antibody mimetics.

In the context of the invention, the term “microfluidic device” means a) a device that comprises a plurality of enclosed microchannel structures, each of which comprises one or more enclosed microchannels and/or microcavities, and b) that these microchannel structures are used for transporting and processing liquid aliquots that are in the microliter range and may contain reactants including e.g., analytes and reagents. The liquid aliquots are typically aqueous. The transporting and processing are typically part of an analytically and/or a preparative process protocol. The number of microchannel structures in a device may be ≧5, such as ≧10 or ≧50 and are typically ≦1000, such as ≦500. This kind of devices typically is disc-like, preferably with an axis of symmetry (Cn where n is an integer 2, 3, 4, 5, 6 . . . ∞) perpendicular to the plane of the disc. Disc-like microfluidic devices having this symmetry feature may have rectangular shape, such as squaric shape, and other polygonal shapes for which this symmetry apply. A certain variant is the circular format (n=∞). In particular disc-like devices of the types mentioned may be spun around the axis of symmetry in order to transport liquids within the microchannel structures by the use of centrifugal force. The spin axis does not need to coincide with the axis of symmetry and may or may not intersect the disc plane. The liquid aliquots are typically aqueous and thus include water and mixtures of water with water-miscible organic solvents.

In the context of the invention, the term “microchannel structure” typically comprises microchannels with depths and/or widths that are ≦1,000 μm, such as ≦500 μm ≦200 μm or ≦100 μm or ≦50 μm. In addition to microchannels for transport of liquids there may also be separate channels that vent to ambient atmosphere, either for inlet or outlet of air. The widths and/or depths of venting channels may be in the same range as the other channels, but many times it may be advantageous to make them more narrow and/or more shallow than the channels used for liquid transportation (e.g., with width and/or depths ≦500 μm ≦200 μm or ≦100 μm or ≦50 μm). Yet further, a microchannel structure may comprise one or more of the following inlet port, volume-defining structure, distribution manifold, mixing chamber, reaction microcavity or chamber, detection microcavity or chamber, liquid router, waste outlet, a separation chamber, etc.

The term “microliter range” means liquid aliquots ≦1000 μl, i.e., the range includes the nanoliter range (≦1000 nl) as well as the picoliter range (≦1000 pl).

All patent publications cited herein including corresponding US patents and patent applications are hereby incorporated in their entirety by reference.

### II. The Invention

The present invention suggests a solution to problems associated with the concept of integrating different formats in the same process stream. It is thus a method for the quantification of the amount of each of a plurality of n different analytes (Ans) in one or more liquid samples (analyte samples) by the use of a plurality of n different affinity assay formats, preferably with one separate format dedicated for each analyte. n is an integer ≧2 and typically ≦15, such as ≦10 or ≦5 or ≦4. The individual analytes will be represented by analayte¹, analyte² . . . analyte^{k} . . . analyteⁿ, where analyte^{k} represents any of the analytes if not otherwise apparent from the context.

By proper selection of formats as illustrated in this specification it will be possible to assay the same analyte sample for n selected analytes in the same process stream even if they require different assay formats. The degree of parallelism can be high between the different formats for one, two or more of the affinity reactions that are performed in order to form the affinity complex to be measured and related to the amount of an analyte in a sample. This parallelism can typically also be combined with parallelism for washing steps, diluting steps etc between the formats. Selection of affinity immobilization for the introduction of immobilized reactants (=capturer) will make it easy to adapt capacity and density of capture reactants on a solid phase to the relevant detection range and optimal format of a particular analyte thereby facilitating the possibility of quantifying several different analytes in the same sample by different formats according to the invention. In a preferred variant, a sample containing one or more of the n analytes is used without significant dilution (less than 1:10, such as less 1:5, or even undiluted samples except for a possible removal of particulate matters and other clogging substances) while maintaining a sufficient accuracy and limit of detection in the quantification for all of the selected analytes. In the case of analyte samples from patients and diagnostic variants of the innovative method, it is likely that it will lead at least to the same diagnostic sensitivity as conventional assays in which the n different analytes are quantified in different process streams.

### III. Assay Formats

The term “different formats” contemplates affinity assays that differ from each other with respect to at least one, two, three or more features such as:

(i) the relative order in which the reactants analyte, capturer and detectable reactant (if used) are allowed to react;

(ii) the counterpart relationship between analyte, capturer, and detectable reactant, e.g. if
a) the capturer is or is not an affinity counterpart to the detectable reactant and/or to the analyte;
b) the detectable reactant is or is not an affinity counterpart to the analyte and/or to the capturer;
c) the detectable reactant and the capturer are utilizing the same binding site or two roomly spaced binding sites that may be different or equal on the analyte (=different or equal specificities of the detectable reactant and the capturer for binding the analyte, equal means that the analyte is at least bivalent with respect to the binding site concerned);
d) type of counterpart relationship, for instance if one or more of the analyte, capturer and detectable reactant (if used) is or is not an antibody or an antigen/hapten relative to one or more of the other ones of these reactants;

(iii) general-type of affinity assays, i.e. immunoassay, hybridisation assay etc;

(iv) principle utilized for measuring the product, e.g. detectability due to a separately introduced label or an inherently detectable group, due to an affinity group or a signal-generating group etc;

(v) the state of the solid phase during the capturing of the analyte or a detectable reactant by a solid phase, i.e. porous bed of particles, monolithic porous bed, suspended particles, inner wall of the microcavity etc;

(vi) flow conditions or static conditions during capturing of a reactant such as the analyte or the detectable reactant or when attaching a soluble affinity complex or product to the solid phase;

(vii) number of reaction steps, for instance one-step or two-step format with respect to the use of the analyte, capturer and detectable reactant; or

(viii) limiting or non-limiting amounts/concentrations of the analyte or a counterpart to the analyte.

In a typical affinity assay an uncharacterized amount of an analyte of a sample is allowed to form an affinity complex comprising at least the analyte and an affinity counterpart to the analyte. Depending on the format used, additional affinity reactants may be used and possibly also incorporated into the complex. The amount and type of reactants are selected so that the affinity reactions involved will result in an amount of an affinity complex that will reflect the amount of an analyte in an original or native sample or in the particular sample contacted with one or more of the reactants used.

An assay format used according to the inventive concept utilizes an immobilized or immobilizable form of the capturer and possibly also a detectable reactant. These two reactants may or may not be the same. Either one or both of the capturer or the detectable reactant will be fully or partly incorporated into an affinity complex the amount of which will reflect the amount of analyte in the sample. The analyte may or may not be part of the complex. Immobilization to a solid phase is used in order to facilitate separate measurement of the portion of the detectable reactant that is incorporated into an affinity complex that reflects the amount of analyte in a sample without disturbing influence from the portion of the detectable reactant that is not incorporated.

An affinity assay/format can be classified with respect to the number of incubations (steps) that is required to form the affinity complex to be measured. A single incubation/step in this context means separately reacting an affinity reactant with a previously formed complex or a single reactant (affinity counterpart to the added reactant). In preferred variants of the invention at least two, three, four or more up to all of the n different assay formats have the same number of incubation steps involving one, two, or three of the analyte, the detectable reactant (if present), and the capturer, i.e. is a one-step or a two-step format. Each of these formats may use additional reactants and incubation steps.

One-step formats are called “simultaneous”, i.e. the analyte, the capturer and the analyte counterpart are reacted in the same incubation/step. Two-step formats are called “sequential” formats. Two-step formats in which the first step comprises reaction with the immobilized or immobilizable capturer are “forward”. If the capturer is not involved in the first step the formats are “reversed”. Forward formats are normally preferred to reversed formats.

For one-step and two-step formats that are used in the invention the amount of analyte is determined from the amount complex (product) on the solid phase preferably by measuring the detectable reactant on the solid phase. In certain variants measurement may be of the detectable reactant remaining in the liquid after the second step in the two-step variant and after the single step in the one-step variant.

A. Inhibition Formats

In inhibition (=competitive) formats an analyte and an analyte analogue are competing with each other for binding to an affinity counterpart to the analyte. The counterpart is typically in a limiting amount. Amount in this context may include concentration.

An inhibition format as used in the invention, typically utilizes an analyte counterpart that is:

A) immobilized or immobilizable (=the capturer) if the analyte analogue is soluble and detectable (=the detectable reactant), and

B) detectable (=the detectable reactant) if the analyte analogue is immobilized or immobilizable (=the capturer).

In preferred variants the desired complex may be accomplished within a microfluidic device from the analyte, the detectable reactant and the capturer by two sequential incubations (two-step format) or by a single incubation with all three reactants simultaneously present (one-step format) possibly followed by an immobilization incubation if the complex formed is immobilizable and comprises an immobilizing tag (one-step format followed by an incubation step for immobilization). If additional reactants are used additional steps may be necessary.

A typical two-step format of variant (B) comprises a first step in which an immobilized or immobilizable analyte counterpart (capturer), e.g. anti-An, is reacted with a) the analyte or b) the detectable analyte analogue, followed by a second step which comprises reaction of the complex formed with the remaining one of the analyte and the detectable analyte analogue. In alternative (a), the second step typically comprises that residual binding sites on the analyte counterpart (capturer) after the first step is estimated by reaction with the detectable analyte analogue (“titration”). In alternative (b) the first step in alternative (b) typically means substantial saturation of the binding sites of the analyte counterpart (capturer) with the detectable analyte analogue while the second step typically comprises displacement of detectable analyte analogue by the analyte from the complex formed in the first step.

A typical two-step format of variant (B) comprises a first step in which the analyte and the detectable analyte counterpart, e.g. anti-An, is reacted with each other in solution followed by a second step comprising capturing of analyte counterpart that has free binding sites for the analyte (uncomplexed counterpart) by a solid phase to which analyte analogue (capturer) is immobilized.

A typical one-step format of variant (A) preferably requires mixing of analyte and detectable analyte analogue upstream or within the microcavity containing the solid phase before reaction with the analyte counterpart (capturer), such as anti-An, immobilized to the solid phase.

Formats that utilize an immobilizable capturer, e.g. an anti-analyte or analyte analogue, comprises in principle the same steps as corresponding formats in which the corresponding reactant is preimmobilized (except that the reaction is taking place in solution). Attachment of an immobilizable reactant (capturer) to the solid phase then typically takes place subsequent to a step during which the first complex that comprises the immobilizable reactant is formed.

The inhibition formats are typically used for analytes that have a low molecular weight and/or are monovalent with respect to binding the analyte counterpart. Thus an analyte in an inhibition format typically has a molecular weight that is relatively low, such as ≦50 000 daltons or ≦10 000 daltons or ≦5 000 daltons or ≦1 000 daltons. The analyte may have polymeric structure, such as biopolymeric structure, as described for reactants in general elsewhere in this specification, but may more often have non-polymeric structure or a low number of repetitive units such as different and/or equal amino acid residues, nucleotides or monosacharide units. A low number in this context is ≦500, such as ≦1000 or ≦100 or ≦50.

B. Non-Inhibition Formats

Non-inhibition formats (=non-competitive formats) typically utilize non-limiting amounts of one or more affinity counterparts to the analyte, typically anti-Ans. For certain variants limiting amounts of the counterparts amounts may be used. Amount in this context includes concentration.

The most important non-competitive formats are of the sandwich-type and comprise formation of immobilized or immobilizable complexes in which an analyte is sandwiched between two analyte counterparts, e.g. two anti-Ans, that are directed towards binding sites that are remotely spaced on the same analyte molecule (i.e. allow that both anti-analytes can be bound simultaneously to the analyte). Typically, one of the analyte counterparts is the detectable reactant and the other one is the immobilized or immobilizable capturer. The binding sites on the analyte are typically different which implies that the two counterparts used have different specificities. The binding sites involved may in some variants be equal (repetitive, at least bivalent with respect to these binding sites) in the sense that the two counterparts used may react interchangeable with any one of the sites, which also implies that the two counterparts have essentially the same binding specificity.

In a similar manner as for inhibition formats there are two-step formats (sequential formats) and one step formats (simultaneous formats).

Two main two-step variants are: a) the forward format in which the first step comprises incubation of an immobilized or immobilizable analyte counterpart (capturer) with the analyte followed by a second step that comprises incubation of the complex formed in the first step with a second analyte counterpart (the detectable reactant), and b) the reverse format in which the first step comprises incubation of a soluble detectable analyte counterpart (the detectable reactant) with the analyte followed by a second step that comprises incubation of the complex formed in the first step with an immobilized or immobilizable analyte counterpart (capturer).

A simultaneous one-step format of a sandwich assay comprises incubating an immobilized or immobilizable analyte counterpart (capturer), a soluble detectable analyte counterpart (the detectable reactant) and the analyte for the formation of an at least ternary complex that comprises all three of the reactants without separate preformation of a binary complex that comprises only two of the reactants.

The forward format is preferred in the invention, in particular the variant utilizing an immobilized analyte counterpart (capturer).

In a sandwich format the amount of analyte is preferably determined from the amount complex (the product) on the solid phase preferably by measuring the detectable reactant on the solid phase after the second step in the two-step variant and after the single step in the one-step variant. In principle measurement of the detectable analyte counterpart (detectable reactant) remaining in the liquid after formation of the product may be feasible.

An analyte in a sandwich assays is at least bivalent (=polyvalent) and has a relatively high molecular weight in order to permit simultaneously binding of two anti-analytes. The molecular weight thus may be ≧5 000 daltons, such as ≧10 000 daltons or ≧50 000 daltons. This kind of analytes typically comprises a polymeric structure, such as biopolymeric structure, as generally described for reactants elsewhere in this specification. The number of subunits such as different and/or equal amino acid residues, nucleotides, monosaccharide units etc typically is ≧50, such as ≧100 or ≧500.

A sandwich format that utilizes two analyte counterparts (as the capturer and the detectable reactant) that have essentially the same binding specificity are particular adapted for the assay of analytes that are least bivalent with respect to the binding site utilized on the analyte. One important type of analytes that complies with this condition is an at least bivalent antibodies and the assay formats concerned may thus be used for the assay of antigen-specific antibodies irrespective of class, subclass or species. In this kind of sandwich format, limiting amounts and/or selected densities of the capturer are typically advantageous in order to secure simultaneous binding of the two counterparts to the same analyte molecule. This in particular applies to the counterpart bound to the solid phase (capturer). Careful selection of the amount of the detectable counterpart is also appropriate in order to avoid disturbing formation of soluble ternary complexes comprising the analyte sandwiched between two detectable analyte counterparts. For more details see PCT/SE2006/000071 (Gyros Patent AB).

Sandwich formats that utilize two analyte counterparts, such as anti-An₁ and anti-An₂, (as the capturer and the detectable reactant), that have different binding specificities are in particular adapted for analytes that expose two different binding sites that are roomly spaced. This kind of formats can in principle be used for measuring any larger biomolecule that is polyvalent and comprises one or more of the structures given below for reactants in general. The format is particularly well-adapted for measuring a particular subpopulation of a group of substances where each subpopulation has a) a binding site (common binding site) that also is present in the other subpopulations, and b) another different binding site that does not exist in any of the other subpopulations. The sandwich format referred to in this paragraph can be illustrated with the assay of A) an antigen-specific antibody of a certain Ig-class, Ig-subclass, species etc (one counterpart is an antigen/hapten and the other is an anti-Ig-class antibody, anti-Ig-subclass antibody, or some other Ig-binding reactant), B) an Ig-subclass or Ig-class, such as IgA, IgD, IgE, IgG, IgM (e.g. two different anti-Ig antibodies), and individual variants of other substances that are more or less polymorphic and/or comprises isoforms and/or only comprises two roomly spaced binding sites that are different.

Another non-competitive variant utilizes only one analyte counterpart, which is in immobilized or immobilizable form. In this case complex formation leads to an immobilized complex, or a soluble complex that subsequently is immobilized. The immobilized complex as such is then measured. This kind of non-inhibition formats is typically of the one-step type. The immobilized or immobilizable counterpart may be detectable, e.g. comprise a signal-generating label for which the signal is changed as a consequence of binding to the analyte (coinciding capturer and detectable reactant).

C. Reactants

An individual reactant used is typically selected among members of ligand-receptor pairs, such as a) antigens/haptens, b) antibodies or antigen/hapten-binding fragments thereof including affinity reactants mimicking the antigen/hapten-binding ability of antibodies and their antigen/hapten-binding fragments, c) nucleic acids including single and double stranded forms, and polynucleotides and oligonucleotides and mimetics of nucleic acids, d) hormones, such as of steroid structure or peptide structure and hormone receptors, and e) components of catalytic systems, such as biocatalytic systems like enzymatic systems.

An affinity reactant used in the context of the invention typically exhibits one or more structures selected among members of the group consisting of:

A) amino acid structures including protein structures such as peptide structures such as poly and oligopeptide structures, and including mimetics and chemically modified forms of these structures etc;

B) carbohydrate sugar structures, such as polysaccharide structure including monosacharide and oligosaccharide structure, including mimetics and chemically modified forms of these structures, etc, and other sugar structures;

C) nucleotide structures including nucleic acid structures, and mimetics and chemically modified variants of these nucleotide structures, etc;

D) lipid structures such as steroid structures, triglyceride structures, etc, and including mimetics and chemically modified forms of these structures;

E) other structures of organic or bio-organic nature including drugs.

### IV. Microchannel Structure and the Solid Phase (Step (i))

A microchannel structure of a microfluidic device for use in the invention comprises a system of microconduits and microcavities that enables the steps of an assay format that are to be carried out in the structure. Typical microfluidic devices and microchannel structures with different fluidic functionalities have for instance been described by Gyros AB/Amersham Biosciences (WO 99055827, WO 99058245, WO 02074438, WO 02075312, WO 03018198, WO 04103890, WO 05032999, WO 05094976, WO 05072872, PCT/SE2005/001887); Tecan/Gamera Bioscience (WO 01087487, WO 01087486, WO 00079285, WO 00078455, WO 00069560, WO 98007019, WO 98053311); Åmic AB (WO 03024597, WO 04104585, WO 03101424 etc) etc. Included in this list are corresponding issued US patents and published US patent applications.

The microcavity (114a-h) that contains the solid phase typically has at least one cross-sectional dimension that is ≦1 000 μm, such as ≦500 μm or ≦200 μm (depth and/or width). The total volume of the microcavity is typically in the lower μL-range, such as ≦20 μL, such as ≦10 μL or ≦5 μL or ≦1 μL or ≦500 nL.

The microcavity that contains the solid phase may be associated with one, two or more mixing functions that are positioned upstream of the microcavity or at least one of them is partly or fully coinciding with the microcavity. Between two mixing functions or between a mixing function or the microcavity that contains the solid phase there may be an extra microcavity in which the mixed reactants can be incubated and reacted with each other before the reaction mixture enters the microcavity containing the solid phase. The preferred design will depend on the physical state of the reactants (soluble or insoluble, soluble includes suspended) that are to participate in the actual reaction step performed in a microcavity. Two examples are:

A step that comprises reaction between a single soluble reactant and a non-suspended solid phase (inner wall and porous bed) does not require any mixing and can thus be carried out with or without the kind of mixing function referred to. Typical formats that comprise this kind of step are forward sequential assays that in the first step involve incubation of a soluble reactant (analyte or detectable reactant) with a capture reactant.

A step that comprises reaction between a mixture of soluble reactants and a reactant on the solid phase requires a mixing function in association with the microcavity containing the solid phase unless the two reactants are mixed outside the microfluidic device. If the soluble reactants are to react with each before contacting the solid phase, there is preferably also an extra microcavity between the mixing function and the microcavity containing the solid phase.

The need for mixing functions and or extra reaction microcavities is apparent from the different formats discussed in detail elsewhere in this specification.

Microchannel structures with mixing functions and incubation microcavities as described in the previous paragraph and their use in affinity assays in which immobilzable reactants are used for the formation of immobilizable affinity complexes that subsequently are immobilized to a solid phase are disclosed in PCT/SE2005/001887 (Gyros Patent AB) and corresponding regular US application “Microfluidic assays and microfluidic devices” filed in December 2005. See also WO 02075312 (Gyros AB).

The solid phase is typically in the form of A) a porous bed, for instance a packed bed of particles or a porous monolith, or B) the inner wall of the microcavity, or C) suspended particles that are capable of sedimenting to a porous bed. In the case of suspended particles or a porous bed comprising suspensible particles, the form of the solid phase can be changed during an assay from a porous bed to suspended particles and vice versa. For instance the change may occur between the first and second step in a sequential assay, immediately before or after the single step in simultaneous assay, before or after an immobilization step, before or after a washing step etc. Any one of these steps may thus take place with a particulate solid phase in suspended form or in bed form while the preceding or subsequent step is taking place with either of the two forms. One or more external magnets in combination with centrifugal force can support the change between the forms if the particles contain a magnetic material. See for instance M Gruman et al., 8^{th} International Conference on Miniaturized Systems for Chemistry and Life Sciences (Malmo, Sweden), Sep. 25-30, 2004, pp. 593-595 and Steigert et al., J. Assoc. Lab. Autom. October (2005), 331-341. See also SE 0600557-3 and corresponding US provisional application filed in parallel on Mar. 13, 2006 (“Enhanced magnetic particle stirring”, Per Andersson and Gérald Jesson, Gyros Patent AB) which both hereby are incorporated by reference in their entirety.

The microcavity (114a-h) typically has a certain region (104a-h) to which the solid phase can be located, for instance as a porous bed. This region typically is associated with an outlet that is in fluid communication with downstream parts of a microchannel structure permitting selective downstream transport of liquid and/or the particles. A porous bed typically occupies a volume in the nL-range, such as ≦5 000 nL, such as 1 000 nL or ≦500 nL ≦100 nL or ≦50 nL or ≦25 nL

Suitable particles for solid phases are preferably spherical or spheroidal (beads), or non-spherical. Appropriate mean diameters for particles are typically found in the interval of 1-100 μm with preference for mean diameters that are ≧5 μm, such as ≧10 μm or ≧15 μm and/or ≦50 μm. Also smaller particles can be used, for instance with mean diameters down to 0.1 μm. Diameters refer to the “hydrodynamic” diameters. Particles to be used may be monodisperse (monosized) or polydisperse (polysized) in the same meaning as in WO 02075312 (Gyros AB).

The base material of a solid phase may be made of inorganic and/or organic material. Typical inorganic materials comprise glass. Typical organic materials comprise organic polymers. Polymeric materials comprise inorganic polymers, such as glass and silicone rubber, and organic polymers of synthetic or biological origin (biopolymers). The term “biopolymer” includes semi-synthetic polymers in which there is a polymer backbone derived from a native biopolymer. Appropriate synthetic organic polymers are typically cross-linked and are often obtained by the polymerisation of monomers comprising polymerisable carbon-carbon double bonds. Examples of suitable monomers are hydroxy alkyl acrylates, for instance 2-hydroxyalkyl acrylates such as 2-hydroxyethyl acrylates, and corresponding methacrylates, acryl amides and methacrylamides, vinyl and styryl ethers, alkene substituted polyhydroxy polymers, styrene, etc. Typical biopolymers in most cases exhibit carbohydrate structure, e.g. agarose, dextran, starch etc.

The particles of solid phases may be manufactured from non-magnetic material, e.g. polymeric, into which minor particles of magnetic material, such as ferrite, have been incorporated, or the particles may be based on magnetic particulate material, such as ferrite, that may have been appropriately surface modified.

The solid phases used in the invention are preferably hydrophilic. For porous beds this means that surfaces of the pores of a bed has a sufficient wettability for water to be spread by capillarity all throughout the bed when in contact with excess water (absorption). If the solid phase is the inner wall of the region (104a-h) where the solid phase is placed, the term “hydrophilic” primarily contemplates that the water contact angle of the inner surfaces at this location is within the limits specified for hydrophilicity (wettability) elsewhere in this specification. Alternatively the hydrophilicity is sufficient to fill the location (104a-h) with water by capillarity once water has reached the most upstream end of it. Surfaces that are to be in contact with aqueous liquids shall also expose a plurality of polar functional groups which each has a heteroatom selected amongst oxygen and nitrogen, for instance. Appropriate functional groups can be selected amongst hydroxy groups, eythylene oxide groups (—X-[CH₂CH₂O—]ₙ where n is an integer >1 and X is nitrogen or oxygen), amino groups, amide groups, ester groups, carboxy groups, sulphone groups etc, with preference for those groups that are essentially uncharged independent of pH, for instance within the interval of 2-12.

If the base material of a solid phase material is hydrophobic or not sufficiently hydrophilic, e.g. is based on a styrene (co)polymer, the surfaces that are to be in contact with an aqueous liquid may be hydrophilized. Typical protocols comprise coating with a compound or mixture of compounds exhibiting polar functional groups of the same type as discussed above, treatment by an oxygen plasma etc.

The technique for introducing an immobilised capturer on the solid phase typically comprises:

A) firmly attaching a soluble form of the capturer to the solid phase, or

B) building an immobilized capturer stepwise on the solid phase (solid phase synthesis).

Both routes are commonly known in the field. The linkage to the solid phase material may be via covalent bonds, affinity bonds (for instance biospecific affinity bonds), physical adsorption, electrostatic bonds etc.

Alternative a) typically makes use of an immobilizing group on the solid phase and an immobilizing tag on the capturer which are mutually reactive with each other to the formation of a bond that resists undesired cleavage under the conditions provided when carrying out the inventive method. The immobilizing group is introduced on the solid phase material before reaction with the immobilizing tag. The immobilizing group and the immobilizing pair defines an immobilizing pair.

Covalent immobilization for variant (a) means that the cleavage-resistant bond is covalent. The immobilizing group and the immobilizing tag are typically selected amongst mutually reactive electrophilic and nucleophilic groups, respectively. Examples of groups are for instance given in WO 2004083109, PCT/SE06/000071, and PCT/SE06/000072 (all Gyros AB/Gyros Patent AB).

Immobilization via affinity bonds may utilize an immobilizing affinity pair in which one of the members (immobilized ligand L=immobilizing group) is firmly attached to the solid phase material while the other member (immobilizing binder, B) is part of a conjugate (immobilizing conjugate) that contains a first moiety that comprises binder B (=immobilizing tag) and a second moiety that comprises a binding site that is capable of affinity binding to the analyte An. The pair is typically selected to be the same in at least two, three, four etc of the n formats/microchannel structures and not to negatively interfere with the desired binding activity of the reactants of these formats/structures. In other words the binder B and the affinity ligand L are generic for these formats/structures. Typical preferred immobilizing affinity pairs are biotin-binding compounds such as streptavidin, avidin, neutravidin, anti-biotin antibodies etc and biotin, b) anti-hapten antibodies and the corresponding haptens or antigens, and c) class/subclass-specific antibodies and Igs from the corresponding class.

The term “conjugate” above and in other contexts of the invention refers to covalent conjugates, such as chemical conjugates and recombinantly produced conjugates. A conjugate comprises at least two moieties bound together, typically covalently, via a linker. The term also includes so-called native conjugates, i.e. affinity reactants which each exhibits two binding sites that are spaced apart from each other and with affinity directed towards two different molecular entities. Native conjugates thus includes an antigen which has physically separated antigenic determinants that are different, an antibody which comprises a species and/or class-specific determinant in one part of the molecule and an antigen/hapten-binding site in another part.

Preferred immobilizing affinity pairs (L and B) typically have affinity constants (KL-B=[L][B]/[L-B]) that are at most equal to the corresponding affinity constant for streptavidin and biotin, or ≦10¹ times or ≦10² times or ≦10³ times larger than this latter affinity constant. This typically will mean affinity constants that roughly are ≦10⁻¹³ mole/l, ≦10⁻¹² mole/l, ≦10⁻¹¹ mole/l and ≦10⁻¹⁰ mole/l, respectively. These affinity constant ranges refer to values obtained by a biosensor (surface plasmon resonance) from Biacore (Uppsala, Sweden), i.e. with the ligand L immobilized to a dextran-coated gold surface.

Ranges for suitable binding capacities for a binder B and measurement of such binding capacities have been given in WO 2004083109, PCT/SE06/000071, and PCT/SE06/000072 (all Gyros AB/Gyros Patent AB).

Immobilizing groups and immobilized capture reactants may be introduced on a solid phase as described in WO 2004083109, PCT/SE06/000071, and PCT/SE06/000072 (all Gyros AB/Gyros Patent AB).

Densities and amounts of immobilized capturer on the solid phase can easily be varied in a controlled manner by attaching a soluble form of the capturer in an inhibition mode. This mode means that a solid phase exhibiting the immobilizing group is contacted with a liquid sample that contains dissolved forms of a capture reactant and a non-sense reactant both of which exhibit an immobilizing tag that is reactive with the immobilizing group. The amount of each of these reactants and/or their total amount shall typically be in excess compared to the amount of immobilizing groups on the solid phase. The actual density of capturer in the final solid phase will then be determined by the relation between the rates of the immobilization reaction of the capturer and the nonsense reactant. In this way it will be simple to adapt the solid phase binding capacity for an affinity counterpart to the capturer and/or density of capturer in each of the n microchannel structures to the actual format and analyte of this structure. This kind of introduction of capturer may alternatively be carried out in a batch mode with subsequent transfer of the solid phase produced to the individual structures of a microfluidic device.

Immobilizing affinity pairs are preferred.

### V. Affinity Complex (Step (ii))

These reactions are performed upstream and/or within the microcavity that contains the solid phase and involve the analyte and the capturer in immobilizable and/or immobilized form to give a product in immobilized form in an amount that is a function of the amount of original or native analyte in the original/native sample. In many formats this also means that the consumption at least one of the reactants used is a function of the amount of the analyte. The product may initially be formed in a soluble but immobilizable form that subsequently is immobilized. A detectable reactant may be included in order to facilitate measurement of the amount of the product (see elsewhere in this specification).

The immobilized/immobilizable feature of the product typically resides in the fact that the capturer is provided in immobilized or immobilizable form in step (i). As discussed above a reactant and/or a product that is immobilizable typically has an immobilizing tag. This tag is reactive with an immobilizing group on the solid phase. See above.

### VI. Determination of the Amount of an Analyte (Step (iii))

In step (iii) the amount of the product formed in the microcavity of a microchannel structure is measured and the value obtained is related to the amount of analyte in an original sample. The measurement may be of the amount of product formed or of the consumption of a reactant involved in the formation of the product, for instance as the remaining amount of this latter reactant.

In order to find the relation between a measured value and amount of analyte known principles are applied, typically by comparing a measured value with corresponding values that have been obtained for one or more standard samples. The comparison typically is with a) a series of one, two or more samples containing varying known amounts of the analyte, b) one or more samples obtained at an earlier occasion for instance from the same or a different individual, c) one or more samples obtained from healthy individuals or from individuals having a particular disease state, etc. The quantification is typically absolute, for instance as a concentration. It may also be relative, e.g. relative to some kind of constituents of the liquid entering the microcavity in which the reaction is taking place or of an original sample, relative to another sample taken at an earlier or a later occasion and/or from the same or another individual etc.

In order to facilitate the measurement in step (iii) of the formation of the product in/on a particular microchannel structure/microcavity/solid phase there is preferably used an analytically detectable reactant that is measurable as such after having been incorporated into the product and/or otherwise enables measurement of the product. The number of analytes/assay formats/microchannel structures that utilizes the detectable reactant is typically ≧1, such as ≧2 or ≧3 or ≧4, and is always ≦n (=the total number of assay formats used for the plurality of n analytes). This does not exclude that detectable reactants may also be used for analytes/formats/microchannel structures that are not included amongst the n analytes. The detectable reactant may in certain formats be the capturer in immobilized or immobilizable form or the analyte.

The detectable reactant may be introduced into the microcavity containing the solid phase a) prior to, b) simultaneous with or c) subsequent to the introduction of the analyte in step (ii) for one, two or more of the analytes/microchannel structures/formats. There is a preference for parallelism between two or more, such as all of the n analytes/microchannel structures/formats for this introduction of the detectable reactant and/or its reaction with an affinity counterpart that may be immobilized or immobilizable to the solid phase, such as the capturer, an affinity complex that comprise the analyte and formed prior to the introduction of the detectable reactant, etc.

Alternative (a) of the preceding paragraph may be used for I) attaching a detectable reactant that also is a capturer to the solid phase, or II) performing an immobilized complex that comprises the immobilized capturer and the detectable reactant (provided the detectable reactant and the capturer are affinity counterparts). Each of these two variants is applicable to one-step and two-step formats as discussed above for inhibition and non-inhibition formats including also sandwich formats.

Alternative (b) may be used in one-step as well as in two-step formats as discussed above for inhibition and non-inhibition formats including also sandwich formats.

Alternative (c) typically comprises forward sequential two-step formats as discussed above for inhibition and non-inhibition formats including also sandwich formats.

Step (i) will be a part of step (ii), if the capturer in immobilizable form is included in a premixture introduced into the microcavity.

In the case a complex between two affinity counterparts is formed in a premixture in an amount, which is a function the amount of the analyte in the original sample, this complex will be an example of an analyte-related entity.

The same detectable reactant or the same capturer may be utilized for two, three or more of the plurality of n analytes/formats/microchannel structures.

A detectable reactant typically comprises a moiety 1 in which there is a detectable group and a moiety 2 which provides the desired reactivity towards an analyte or towards an immobilized or immobilizable capturer. A detectable reactant may thus be a conjugate in which moiety 1 and moiety 2 are firmly attached to each other, preferably by covalent bonds. This conjugate may be native or synthetic. For synthetic conjugates the detectable group will be called “label”.

The detectability of moiety 1 typically resides in the fact that it comprises a group that can be analytically detected and quantified. Signal-generating groups and affinity groups are typical examples. A signal-generating group may be selected amongst radiation emitting or radiation absorbing groups and groups that in other ways interfere with a given radiation. Particular signal-generating groups are enzymatically active groups such as enzymes, cofactors, substrates, coenzymes etc; groups containing particular isotopes such as radioactive or non-radioactive isotopes; fluorescent and fluorogenic groups; luminescent and luminogenic groups including chemiluminescent and chemiluminogenic groups, bioluminescent and bioluminogenic groups etc; metal-containing groups including groups in which the metal is in ionic form etc. Affinity groups in this context are typically detected by the use of a secondary detectable reactant that is a conjugate between an affinity counterpart to the detectable affinity group and a second detectable group that is different from the detectable group in the detectable reactant, and preferably is a signal-generating group typically in the form of a label. Typical affinity based detectable groups may be selected amongst the individual members of the immobilizing affinity pairs discussed elsewhere in this specification, with the proviso that an affinity based detectable group should not be capable of affinity binding during the method to a member of an immobilizing binding pair if such a pair has been used for the immobilization of the capturer to the solid phase.

In other variants, the detectability of a reactant resides in features other than presence of a signal-generating or an affinity group. Detectability/measurability thus may reside in the increase in volume or mass that a reactant adds to the affinity complex formed on the solid phase. The reactant as such in this variant defines the detectable group. See for instance WO 03102559 (Gyros AB).

The actual measurement is typically carried out in the microcavity in which the product is formed in immobilized form. Alternatively, the actual measurement may take place downstream of this microcavity, for instance in a separate detection microcavity. This latter variant may involve measurement of excess of detectable reactant that passes through the microcavity or of a soluble and detectable entity generated from the product formed on the solid phase in step (ii) etc. The capturer may for instance be attached to the solid phase by a cleavable bond. As an alternative the detectable group in the detectable reactant may be bound to other parts of the reactant by a cleavable linker. See for instance U.S. Pat. No. 4,231,999 (Pharmacia Diagnostics AB). After formation of the complex and subsequent washing, if needed, the cleavable linker/bond is splitted, and fragments from the reactant/solid phase and containing the detectable group are transported downstream to a detection microcavity where they are measured. Another alternative is that the detectable group is a reactant in a reaction system that gives rise to a soluble product that can be transported downstream for measurement. Suitable reaction systems enabling the last alternative comprise catalytic systems, such as biocatalytic systems including enzyme systems, in which case the detectable group may be a component of the catalytic system, such as a catalyst, a co-catalyst, a co-factor, a substrate, a co-substrate, an inhibitor, an effector etc. For enzyme systems the relevant components are enzyme, coenzyme, co-factor, substrate, co-substrate, inhibitor, activator, effector etc.

A detectable reactant that contains a first signal-generating group that becomes captured to the solid phase may be combined with a detectable reactant that contains a second signal-generating group that co-operates with the first group to give the appropriate signal when captured to the solid phase. This second group may be selected such that the two groups together give the appropriate signal. This variant may be illustrated with scintillation proximity assays (SPA). The principle with interacting labels may also be illustrated with pairs of fluorophores that may be identical or different and with fluorescence-quencher pairs.

In still another variant the capturer is associated with a signal-generating group for which the signal is changed when the analyte and/or the detectable reactant become captured by the solid phase, for instance as a consequence of the upcoming proximity between the capturer and the analyte/detectable reactant.

### VII. Reactions During Flow Conditions or Static Conditions

The reaction between a soluble reactant and a solid phase that is fixed to a certain region (104a-h) of the microcavity (114a-h) in which the reaction is taking place preferably takes place under flow conditions, i.e. the liquid containing the reactant is continuously flowing through the bed during the reaction. The soluble reactant may be the analyte, the detectable reactant, an immobilizable capturer, an immobilizable complex formed during the assay etc. The solid phase typically exposes an immobilized capturer or an immobilized complex containing the analyte, or simply an immobilizing group. The appropriate flow rate depends on a number of factors, such as a) reactivity of the capturer or of the immobilizing group; b) reactivity of the soluble reactant; c) volume of the porous bed; d) kind of porous bed (the solid phase material, porosity, bed or coated inner wall etc).

It is many times preferred to set the flow rate such that capturing of a through-passing reactant or complex is predominantly occurring in the upstream part of a solid phase, such as a porous bed, that has a fixed position (104a-h) in the microcavity (114a-h), typically with insignificant capturing in the most downstream part, i.e. at the exit end of the solid phase/region.

Typically the flow rate should give a residence time of ≧0.010 seconds such as ≧0.050 sec or ≧0.1 sec for the liquid containing the entity to become captured by the solid phase, such as a porous bed. The upper limit for residence time is typically below 2 hours such as below 1 hour. Illustrative flow rates are within 0.001-10 000 nL/sec, such as 0.01-1 000 nL/sec or 0.01-100 nL/sec or 0.1-10 nL/sec. These flow rate intervals may primarily be useful for solid phase volumes in the range of 1-1 000 nL, such as 1-200 nL or 1-50 nL or 1-25 nL. Residence time refers to the time it takes for a liquid aliquot to pass the solid phase in the reaction microcavity. Optimization typically will require experimental testing for balancing the n microchannel structures/formats/analytes against each other.

The liquid flow through the solid phase can be driven by in principle any kind of forces, e.g. electrokinetically or non-electrokinetically created forces with preference for centrifugal force possibly combined with capillary force for flow paths in microfluidic devices adapted for this. See further below.

### VIII. Samples

The liquid samples transported and processed in a microchannel structure are typically aqueous and may be diluents, wash liquids and/or liquids containing a reactant such as an analyte and/or a reagent, such the immobilized or immobilizable capturer and the detectable reactant. Immobilized reactants that are transported are typically in suspended form, preferably with the reactant immobilized to suspended particles. An analyte sample introduced into a microchannel structure and/or into the microcavity containing the solid phase may be an unprocessed biological fluid sample or may derive from such a fluid. Processing in this context may include a) transforming an original analyte to a form of the analyte as it exists in the sample to be introduced into the microchannel structure or the microcavity containing the solid phase (i.e. transformation to an analyte-related entity), b) diluting, c) removal of cells and/or other particulate material etc. In preferred variants an undiluted original analyte sample as described elsewhere in this specification is used in at least one, two, three, four or more of the n microchannel structures/formats. An undiluted analyte sample may be blood, various liquid blood fractions such as serum or plasma, lachrymal fluid, regurgitated fluid, urine, sweat, cerebrospinal fluid, gastric juice, saliva, lymph or any of the other examples given in the next paragraph.

The term “biological fluid” contemplates any fluid that contains a bio-organic compound that exhibits a structure of the kind indicated above for an analyte, the capturer, and the detectable reactant. In a more narrow sense the same term contemplates a fluid which contains this kind of bio-organic compounds and derives from a fluid, the composition of which at least partially has been determined by living or dead biological material. Typical biological fluids, in particular those from which a sample containing the analyte derives, include cell culture supernatants, tissue homogenates, blood and various blood fractions such as serum or plasma, lachrymal fluid, regurgitated fluid, urine, sweat, semen, cerebrospinal fluid, gastric juice, saliva, lymph, etc as well as various liquid preparations containing a bio-organic compound as discussed above and deriving from these particular biological fluids. For an analyte selected from antibodies, hormones, cell mediators, immune regulatory substances and the like, a liquid sample containing the analyte typically derives from a vertebrate body fluid of the kinds discussed above that contains the analyte. For instance if one of the n analytes is an antibody, hormone, cell mediator, immune regulatory substance etc at least one analyte sample deriving from a selected one of the body fluids given above is used for quantifying all the n analytes. Typical vertebrates are mammals, avians, amphibians, reptiles etc. Typical mammals are whales, humans, mice, rats, guinea pig, horses, cows, pigs, dogs, cats etc. Typical avians are hens, canaries, budgerigars etc. Amongst amphibians and reptiles may be mentioned those that are used as pets or are popular in zoological gardens.

### IX. Microfluidic Devices

A microfluidic device is a device that comprises one, two or more microchannel structures (101a-h) in which one or more liquid aliquots/samples that have volumes in the μL-range, typically in the nanolitre (nL) range transported and/or processed. At least one of these aliquots/samples contains one or more reactants selected amongst analytes and reagents such as capturers and/or the detectable reactants, or soluble products etc and/or buffers and/or the like. The μL-range contemplates volumes ≦1 000 μL, such as ≦100 μL or ≦10 μL and includes the nL-range that has an upper end of 5 000 nL but in most cases relates to volumes ≦1 000 nL, such as ≦500 nL or ≦100 nL. The nL-range includes the picolitre (pL) range. A microchannel structure comprises one or more cavities and/or conduits that have a cross-sectional dimension that is ≦10³ μm, preferably ≦5×10² μm, such as ≦10² μm.

A microchannel structure (101a-h) thus may comprise one, two, three or more functional units selected among: a) inlet arrangements (102,103a-h) comprising for instance an inlet port/inlet opening (105a-b,107a-h), possibly together with a volume-defining unit (106a-h,108a-h) (for metering liquid aliquots to be processed within the device), b) microconduits for liquid transport, c) reaction microcavities (114a-h); d) mixing microcavities/units; e) units for separating particulate matters from liquids (may be present in the inlet arrangement), f) units for separating dissolved or suspended components in the sample from each other, for instance by capillary electrophoresis, chromatography and the like; g) detection microcavities; h) waste conduits/microcavities (112,115a-h); i) valves (109a-h,110a-h); j) vents (116a-i) to ambient atmosphere; liquid splits (liquid routers) etc. A functional unit may have several functionalities, e.g. microcavity (114a-h) may be used both for performing reactions and for measurement/detection.

A microcavity (114a-h) intended for a solid phase in the form of suspended particles or as a porous bed typically comprises a region or other kind of location (104a-h) in which the solid phase can be or is placed prior to, during or subsequent to an intended reaction involving one or more of the reactants used in the format, in particular one or more of the analyte, the capturer and the detectable reactant. This region or location (104a-h) is typically positioned in close association with an outlet end of the microcavity (114a-h).

Various kinds of functional units in microfluidic devices have been described by Gyros AB/Amersham Pharmacia Biotech AB: WO 99055827, WO 99058245, WO 02074438, WO 02075312, WO 03018198, WO 04103890, WO 05032999, WO 05094976, WO 05072872, PCT/SE2005/001887; Tecan/Gamera Bioscience WO 01087487, WO 01087486, WO 00079285, WO 00078455, WO 00069560, WO 98007019, WO 98053311 etc. Included in this list are corresponding issued US patents and published US patent applications.

In advantageous forms a microcavity (114a-h) intended for a hydrophilic porous bed is connected to one or more inlet arrangements (upstream direction) (102,103a-h), each of which comprises an inlet port (105a-b,107a-h) and at least one volume-defining unit (106a-h,108a-h). One kind of inlet arrangement (103a-h) is connected to only one microchannel structure (101a-h) and/or microcavity (114a-h) intended to contain the solid phase material (individual inlet). Another kind of inlet arrangement (102) is common to all or a subset (100) of microchannel structures (101a-h) and/or microcavities (114a-h) intended to contain the solid phase material. The latter variant comprises a common inlet port (105a-b) that typically is combined with a distribution manifold that has one volume-defining unit/volume-metering microcavity (106a-h) for each microchannel structure/microcavity (101a-h/114a-h) of the subset (100). In both variants, each of the volume-defining units (106a-h,108a-h) including their volume-metering microcavities (106a-h,113a-h) in turn is communicating with downstream parts of its microchannel structure (101a-h), e.g. the microcavity (114a-h). Each volume-defining unit/volume-metering microcavity (106a-h,108a-h/106a-h,113a-h) typically has a valve (109a-h,110a-h) at its outlet end. This valve is typically passive, for instance utilizing a change in chemical surface characteristics at the outlet end, such as a boundary between a hydrophilic and hydrophobic surface (hydrophobic surface break) (WO 99058245, WO 2004103890, WO 2004103891 and U.S. Ser. No. 10/849,321 (Amersham Pharmacia Biotech AB and Gyros AB)) and/or in geometric/physical surface characteristics (WO 98007019 (Gamera)).

The volumes that are to be defined/metered are volumes of liquid aliquots to be transported and processed further downstream in the microchannel structures.

Typical inlet arrangements with inlet ports, volume-defining units, distribution manifolds, valves etc have been presented in WO 02074438, WO 02075312, WO 02075775 and WO 02075776 (all Gyros AB).

Each microchannel structure has at least one inlet opening (105a-b,107a-h) for liquids and at least one outlet opening for excess of air (vents) (116a-i,112) and possibly also for liquids (circles in the waste channel (112)).

The microfludic device used in the invention contains at least the n microchannel structures mentioned above. In total the device typically contains ≧10, e.g. ≧25 or ≧90 or ≧180 or ≧270 or ≧360 microchannel structures.

Different principles may be utilized for transporting the liquid within the microfluidic device/microchannel structures between two or more of the functional units. Inertia force may be used, for instance by spinning the disc as discussed in the subsequent paragraph. Other useful forces are capillary forces, electrokinetic forces, non-electrokinetic forces such as capillary forces, hydrostatic pressure etc.

The microfluidic device typically is in the form of a disc. The preferred formats have an axis of symmetry (Cₙ) that is perpendicular to or coincides with the disc plane, where n is an integer ≧2, 3, 4 or 5, preferably ∞ (C_{∞}). In other words the disc may be rectangular, such as square-shaped and other polygonal forms but is preferably circular. Spinning the device around a spin axis that typically is perpendicular or parallel to the disc plane may create the necessary centrifugal force. Variants in which the spin axis is not perpendicular to a disc plane are given in WO 04050247 (Gyros AB).

The preferred devices are typically disc-shaped with sizes and/or forms similar to the conventional CD-format, e.g. sizes that are in the interval from 10% up to 300% of a circular disc with the conventional CD-diameter (12 cm).

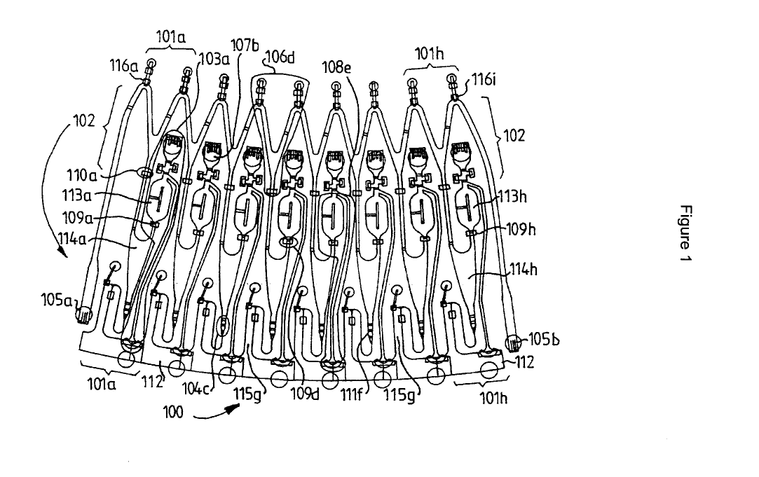

The terms “wettable” (hydrophilic) and “non-wettable” (hydrophobic) of inner surfaces in a microchannel structure contemplate that a surface has a water contact angle ≦90° or ≧90°, respectively. In order to facilitate efficient transport of a liquid between different functional parts of a microchannel structure, inner surfaces of the individual parts should primarily be wettable, preferably with a contact angle ≦60° such as ≦50° or ≦40° or ≦30° or ≦200. These wettability values apply for at least one, two, three or four of the inner walls of a microconduit. In the case one or more of the inner walls have a higher water contact angle, for instance is hydrophobic, this can be compensated for by a more wettable surfaces of one or more of the other inner wall(s). The wettability, in particular in inlet arrangements should be adapted such that an aqueous liquid to be used will be able to fill up an intended microcavity/microconduit by capillarity (self suction) once the liquid has started to enter the microcavity/microconduit. A hydrophilic inner surface in a microchannel structure may comprise one or more local hydrophobic surface breaks in a hydrophilic inner wall, for instance for introducing a passive valve, an anti-wicking means, a vent solely function as a vent to ambient atmosphere etc (rectangles in FIG. 1). See for instance WO 99058245, WO 02074438, US 20040202579, WO 2004105890, WO 2004103891 (all Gyros AB).

Typical microchannel structures for formats that comprise mixing and/or incubation of soluble reactants (the capturer in immobilizable form, the detectable reactant, the analyte and other reactants) to produce an immobilizable affinity complex in an amount that is a function of the amount of analyte in a sample has been described in PCT/SE2005/001887 (Gyros Patent AB) and corresponding regular US application “Microfluidic assays and microfluidic devices” filed in December 2005. See also WO 02075312 (Gyros AB) (see FIGS. 1 and 2). In this kinds of microchannel structures there is typically a first mixing function upstream of the microcavity containing the solid phase and therebetween possibly a first incubation microcavity that may or may not at least partly coincide with the first mixing function. The mixing function may contain one, two or more inlets depending on the number of liquids and/or reactants that are to be mixed. The mixture obtained is transported downstream to the microcavity containing the solid phase possibly via the first incubation microcavity in which reactants in the mixture can react with each other to form an affinity complex before further transport into the microcavity containing the solid phase where the complex can be immobilized. Upstream of the first mixing function and in fluid communication with one or more of the inlets of this mixing function there may be one or more additional mixing functions each of which may or may not be associated with an incubation microcavity in the same manner as the first mixing function is associated with the first incubation microcavity. There may also be additional mixing functions, possibly combined with incubation microcavities connected to the flow path between the first mixing function and the microcavity containing the solid phase, for instance upstream and/or downstream of the first incubation microcavity. These additional mixing functions/incubation microcavities typically occur as branches of the flow path between the microcavity containing the solid phase and the first mixing function. Inlets of this kind of microchannel structure typically have volume-defining units at their inlets for on-device metering of liquid aliquots to be processed within the structure. A volume-defining unit may be associated with an individual inlet or with an inlet common to two or more structures, such as in a distribution manifold. Compare inlet arrangements (103a-h) and (102), respectively, in FIG. 1.

### X. Example

This example suggests quantifying a panel of four analytes in the serum of a patient with symptoms of asthma (allergen related or endogenous asthma).

Analytes and assay formats

| | | Format (all forward two-steps formats |
|---|---|---|
| Analyte | | utilizing immobilized capturer) |
| 1. | Total IgE | Sandwich format with non-limiting amounts |
| | | of anti-IgEs (immobilized capturer and |
| | | detectable reactant) specific for different |
| | | binding sites in the constant region of IgE. |
| 2. | Specific IgE | Sandwich format with non-limiting amounts |
| | (several allergens) | of immobilized allergen and labeled anti- |
| | | IgE |
| 3. | Blocking antibodies | Sandwich format with limiting amounts of |
| | (several allergens) IgG | immobilized allergen and labeled allergen |
| 4. | Inflammation markers | Inhibition format with immobilized anti- |
| | (such as IL-6, IL-8 | inflamation marker and labeled |
| | etc) | inflammation marker. |

Additional analytes can be added to the panel, for instance prostaglandin E. These additional analytes can be quantified according one or more of the four formats given above or by other formats.

### Microfluidic Device and Instrumentation

The microfluidic device is the same as the one shown WO 04083108 (Gyros AB) and WO 04083109 (Gyros AB). The solid phase is polystyrene particles coated with phenyldextran to which streptavidin had been immobilized and packed to a bed/column in the most downstream regions (104a-h) of the microcavities (114a-h). The instrument used for processing is a Gyrolab Workstation equipped with laser fluorescence detector and the microfluidic disc a Bioaffy CD microlaboratory, both being products of Gyros AB, Uppsala, Sweden.

### Reagent Preparation

Immobilizable capturer: The reagents are biotinylated forms of anti-IgE (for analyte1), allergen/antigen (analyte (for analytes 1 and 2), anti-inflammation marker (for analyte 4) and bovine serum albumin (for non-sense immobilized reactant). Biotinylation and work up is carried out essentially as described for the biotinylated reagents in WO 0483108, WO 0483109, PCT/SE2006/000071 (all of Gyros AB/Gyros Patent AB) with due care taken for removal of excess of biotin.

Detectable reactant: The reagents are fluorophor labelled forms of anti-IgE (for analyte 1), allergen/antigen (for analytes 2 and 3) and inflammation marker (for analyte 4). Labelling with Alexa fluorophor 647 and work up is carried out essentially as described for labelling in WO 0483108, WO 0483109, PCT/SE2006/000071 (all of Gyros AB/Gyros Patent AB) with due care taken for removal excess of fluorophor. A polymeric carrier is most likely preferred for low molecular weight analytes, such as an inflammation marker, is preferably used as outlined in PCT/SE2005/001887 (Gyros AB/Gyros Patent AB)

Optimization of limiting amounts of immobilized capturer: Competition conditions with BSA-biotin as the competitor essentially as described in PCT/SE2006/000071 (Gyros Patent AB).

### Assay Procedure

Activation step 1: The capturer is immobilized on the columns (104a-h) in the microcavities (114a-h) by introduction of the biotinylated reagents into the individual inlet ports (107a-h) (excess biotinylated anti-IgE into 107a-b, excess biotinylated allergen/antigen into 107c-d, an optimized mixture biotinylated allergen/antigen and biotinylated BSA (excess of biotin) into 107e-f, and an optimized mixture of biotinylated inflammation marker and biotinylated BSA (biotin in excess) into 107g-h. Subsequently the disc is spinned thereby forcing the reagents to pass into the microcavities (114a-h) and through the solid phases (104a-h) thereby introducing the capturer on each of them.

Step 1: Capture of analyte. An undiluted serum sample is introduced into the common inlet port thereby filling up the volume-defining units (106a-h) in the distribution manifold. Subsequently the disc is spinned thereby forcing the liquid in each volume-defining unit to pass into its downstream microcavity (114a-h) and through the corresponding column (104a-h) where the analyte is captured.

Step 2: Measuring of the amount of analyte captured in step 2. The fluorophor labeled reactants are introduced via the individual inlet ports (107a-h) with fluorophor labeled anti-IgE via ports (107a-b), fluorophor labeled allergen/antigen via ports (107c-f), and fluorophor labeled inflammation marker via ports (107g-h). The fluorescence from the columns is measured with three different PMT settings before introduction of fluorophor labeled reagents and after the introduction plus washing. There should also be separate wash steps before, between, and after each addition of liquids containing reagents or analytes. Se the spin protocol given in the next paragraph.

The spin steps and detection/measuring steps could be:

Initial needle wash: Particle wash 1, Particle wash spin 1, Particle wash 2 structure, Particle wash 2 common, and Particle wash spin 2.

Capture reagent addition structure: Capture reagent spin, Capture reagent wash 1.

Analyte addition common: Analyte spin, Analyte wash 1, Analyte wash spin 1, Analyte wash 2, and Analyte wash spin 2.

CD alignment 1: Detect background PMT 1%, Detect background PMT 5% and Detect background PMT 25%, Spin out.

Detection reagent addition structure: Detection reagent spin, Detection reagent wash 1, Detection reagent wash spin 1, Detection reagent wash 2, Detection reagent wash spin 2, Detection reagent wash 3, Detection reagent wash spin 3, Detection reagent wash 4, Detection reagent wash spin 4.

CD alignment 2: Detect PMT 1%, Detect PMT 5%, Detect PMT 25%

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for quantifying n different analytes that are present in one or more liquid samples by performing n different affinity assay formats, each of which is dedicated for one analyte and results in an affinity complex that is related to the amount of analyte to which the format is dedicated, said method comprises the steps of:
performing each of n different affinity assay formats in a separate microchannel structure of a microfluidic device that contains at least n microchannel structures, and
forming and measuring the affinity complex that is formed on a solid phase that has been placed in a microcavity of the microchannel structure used for the format in order to quantify the analyte to which the format is dedicated.

2. The method of claim 1, wherein each of the n formats comprises the steps of:
(i) providing in a microcavity of the microchannel structure used for the format a solid phase that exposes:
a) an immobilized affinity capturer, or
b) an immobilizing group that is capable of attaching an immobilizable affinity capturer having an immobilizing tag that is reactive with the immobilizing group,
(ii) forming an immobilized form of the affinity complex within the microcavity
a) performing the affinity reaction(s) of the format to incorporate the immobilized capturer of the solid phase provided in step (i.a) into an immobilized form of said complex, or
b) performing the affinity reaction(s) of the format to incorporate the immobilizable capturer into an immobilizable form of said complex and subsequently attaching this complex to the solid phase by reacting the immobilizing tag with the immobilizing group of the solid phase provided in step (i.b), and
(iii) determining the amount of analyte in the sample by measuring the amount of the immobilized complex formed in step (ii).

3. The method of claim 2, wherein the solid phase is in the form of a porous bed during step (ii) for at least one of said n formats.

4. The method of claim 2, wherein at least one of said n formats utilizes a detectable reactant that is an affinity counterpart to the capturer or to the analyte.

5. The method of claim 4, wherein one or more of said at least one formats comprise an inhibition format comprising:
the detectable reactant is an An-analogue, and
the capturer is a counterpart to the analyte and the An-analogue and is immobilized to the solid phase, and said complex comprises the capturer bound to the detectable reactant and/or to the analyte.

6. The method of claim 4, wherein one or more of said at least one formats comprise an inhibition format comprising:
the detectable reactant is a counterpart to the analyte, and
the capturer is an An-analogue and preferably is immobilized to the solid phase, and
said complex comprises the capturer bound to the detectable reactant.

7. The method of claim 4, wherein one or more of said at least one formats comprise a sandwich format having the capturer immobilized to the solid phase comprising:
the detectable reactant and the capturer are counterparts to the analyte,
the analyte comprises two binding sites permitting simultaneous binding of both the detectable reactant and the capturer, and
said complex comprises the analyte bound to both the capturer and the detectable reactant.

8. The method of claim 4, wherein one or more of said at least one formats comprise a sandwich format having an antigen-specific antibody assay and the capturer is immobilized to the solid phase comprising:
the detectable reactant and the capturer are counterparts to the analyte,
the analyte is bivalent with respect to a binding site for which both the detectable reactant and the capturer have specificity, and
said product comprises the analyte bound to both the detectable reactant and the capturer.

9. The method of claim 4, wherein one or more of said at least one formats comprises a sandwich format having an antigen-specific antibody assay and using the capturer immobilized to the solid phase comprising:
the detectable reactant and the capturer are counterparts to the analyte and have binding specificities to two different sites on the analyte,
the analyte comprises said two different binding sites, and
said complex comprises the analyte bound to both the detectable reactant and the capturer.

10. The method of claim 1, wherein at least one of said n formats is an immunoassay.

11. The method of claim 1, wherein at least one of said n formats is an inhibition format.

12. The method of claim 1, wherein at least one of said n formats is a non-inhibition format.

13. The method of claim 1, wherein at least one of said n formats is a sandwich format.

14. The method of claim 2, wherein step (i) comprises providing the capturer in immobilized form for at least one of said n formats.

15. The method of claim 4, wherein for at least two, or more of said n formats comprises that the capturer provided in step (i) is in immobilized form, and two-step sequential formats.

16. The method of claim 1, wherein at least one of said n formats the capturer is immobilized to the solid phase via a generic immobilizing affinity pair.

17. The method of claim 1, wherein at least one of said n formats the capturer is or has been immobilized to the solid phase via a generic immobilizing affinity pair during competition with a nonsense reactant that is capable of becoming immobilized via the same immobilizing affinity pair as the capturer.

18. The method claim 1 and at least one of said n formats is an inhibition format, and at least one of said n formats is a non-inhibition format.

19. The method of claims 1, further comprising
performing at least two of said n formats in a set of microchannel structures that have a distribution manifold in common and each microchannel structure comprises one separate volume-metering microcavity; and
passing for each structure of the set a sub-aliquot 1 that derives from one common liquid sample into the microcavity containing the solid phase by performing the sub-steps of:
(i) providing an aliquot of said common liquid sample in the distribution manifold, and dividing the sample into sub-aliquots with one sub-aliquot per structure of the set,
(ii) processing including transporting a sub-aliquot to provide sub-aliquot 1 at the inlet end of the microcavity containing the solid phase, and
(iii) passing sub-aliquot 1 into the microcavity.

20. The method of claims 1, further comprising
performing at least one of said n formats in a microchannel structure having an inlet port which is not common with the inlet port of the microchannel structures that are used for the other ones of said at least one format, and in the downstream direction is in flow communication with the microcavity containing the solid phase, possibly with a volume-metering microcavity between the microcavity containing the solid phase and the inlet port, and
passing a sub-aliquot 2 deriving from a liquid sample into the microcavity containing the solid phase by the sub-steps of:
(i) providing an aliquot of the liquid sample at said inlet port,
(ii) processing including transporting said aliquot to provide sub-aliquot 2 at the inlet of the microcavity containing the solid phase, and
(iii) passing sub-aliquot 2 into the microcavity.
